(19) United States
(12) Patent Application Publication
(10) Pub. No.: US 2006/0235386 A1
(43) Pub. Date: Oct. 19, 2006

(54) Magnetic manipulation of a cable in blind approach

(75) Inventor: Kent M. Anderson, Memphis, TN (US)

Correspondence Address: KRIEG DEVAULT LLP, ONE INDIANA SQUARE, SUITE 2800, INDIANAPOLIS, IN, 46204-2709, US

(73) Assignee: SDGI Holdings, Inc.

(21) Appl. No.: 11/105,993

(22) Filed: Apr. 14, 2005

(51) Int. Cl. A61F 2/30

(52) U.S. Cl. 606/61

(57) ABSTRACT

One embodiment of the present application is directed to a method and system for performing a unilateral surgical procedure to implant a prosthetic device between two spinous processes. During this procedure at least one side of one of the processes remains covered by soft tissue. An instrument is magnetically coupled to cabling that is routed beneath this soft tissue and remains visually hidden during such routing. The cabling is also configured to engage the prosthetic device positioned between the spinous processes.

### BACKGROUND

The present invention relates to prosthetic apparatus, and more particularly, but not exclusively, relates to an instrument and process for manipulating cabling of such apparatus that may be visually hidden during surgery.

The use of prosthetic implants to address orthopedic injuries and ailments has become commonplace. Nonetheless, there is an ever present challenge to enable less invasive surgical techniques, shorten the time required to surgically implant a prosthesis, and/or provide other improvements. Thus, there is a need for additional contributions in this area of technology.

### SUMMARY

One embodiment of the present application is a unique prosthesis. Other embodiments include unique methods, systems, devices, instrumentation, and apparatus involving an implantable prosthesis.

Another embodiment of the present application is a surgical implantation procedure that includes routing a cable through tissue. At least an end portion of the cable is visually hidden beneath tissue during the procedure. An instrument with a hook-shaped structure magnetically couples to the cable end portion to guide it through tissue. One or more of the cable end portion and the hook-shaped structure includes a magnet. As used herein, “cable” and “cabling” each broadly include one or more filaments, wires, cords, tethers, strands, straps, fibers, or the like; and can be solid, porous, tubular, woven, twisted, braided, and/or such other structural arrangement as would occur to those skilled in the art. Further such “cable” or “cabling” can have any composition, including any essentially pure elemental metal, metallic alloy, organic polymer, organometallic, inorganic substance, and/or composites, to name a few.

A further embodiment of the present application includes: performing surgery to implant a prosthesis between two spinous processes, where one of the spinous processes remains covered by soft tissue throughout this surgery; magnetically coupling an instrument to cabling; advancing the coupled cabling through the soft tissue and about one of the spinous processes using the instrument, with at least a portion of the cabling being visually hidden during advancement; and engaging the cabling to the prosthesis.

Still another embodiment includes a surgical instrument to perform a unilateral surgical procedure to implant an interspinous prosthetic device. During this procedure, an underlying tissue region is exposed by surgical incision on a side lateral to a sagittal plane through the patient's spine, while leaving at least a substantial portion of the opposing lateral side intact. The instrument has a longitudinal central portion and a hook-shaped end portion. The central portion extends a greater distance along a longitudinal axis of the instrument than the hook-shaped end portion. The hook-shaped end portion extends away from the longitudinal axis along a plane. This plane subtends an angle with respect to the longitudinal axis along the central portion that is in a range from about 45 degrees through about 135 degrees for a preferred form of this embodiment. In a more preferred form, this range is from about 75 degrees to about 105 degrees. In an even more preferred form, the angle subtended is about 90 degrees. The hook-shaped end portion includes one or more of a magnet and a material attracted to a magnet to magnetically couple with a portion of a cable visually hidden by soft tissue that remains on a lateral side of a spinous process throughout the procedure.

Yet another embodiment is directed to a system that includes a prosthetic device arranged to be implanted between two spinous processes of a spine of a patient, cabling to engage this device and tissue of the patient, and a surgical instrument. This instrument includes a longitudinal central portion and an instrument end portion. The central portion extends a greater distance along the longitudinal axis of the instrument than the instrument end portion. This end portion includes a hook-shaped structure extending along a plane that intersects the longitudinal axis. An angle subtended between the plane and the longitudinal axis along the central portion is in a range from about 45 degrees through about 135 degrees for a preferred form of this embodiment. In a more preferred form, this range is from about 75 degrees to about 105 degrees. In an even more preferred form, the angle subtended is about 90 degrees. The hook-shaped structure includes one or more of a magnet and a material attracted to a magnet to magnetically couple with a portion of the cabling visually hidden by soft tissue.

One object of the present application is to provide a unique prosthesis.

Alternatively or additionally, another object of the present application is to provide a unique prosthetic method, system, device, instrument, kit and/or apparatus.

Further embodiments, forms, features, aspects, benefits, objects, and advantages of the present application shall become apparent from the detailed description and figures provided herewith.

### BRIEF DESCRIPTION OF THE DRAWING

FIG. 1 is a partial diagrammatic top view of prosthetic apparatus during implantation by a unilateral surgical procedure.

FIG. 2 is a partial diagrammatic side view corresponding to view line 2-2 shown in FIG. 1.

FIG. 3 is a partial diagrammatic and cutaway top view of the apparatus of FIG. 1 representative of a different stage of the unilateral surgical procedure represented by FIGS. 1 and 2.

FIG. 4 is a side view of an instrument shown in FIG. 3; where the FIG. 3 view of the instrument corresponds to section line 3-3 shown in FIG. 4.

### DETAILED DESCRIPTION OF SELECTED EMBODIMENTS

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

One embodiment of the present application includes a technique for performing a surgical procedure to implant prosthetic apparatus. The prosthetic apparatus includes a cable. An instrument is utilized during the procedure that has a hook-shaped structure to magnetically couple to an end portion of the cable. While the instrument is coupled to the end portion of the cable, the instrument is moved to advance the cable through tissue with the end portion of the cable being visually hidden beneath at least part of the tissue.

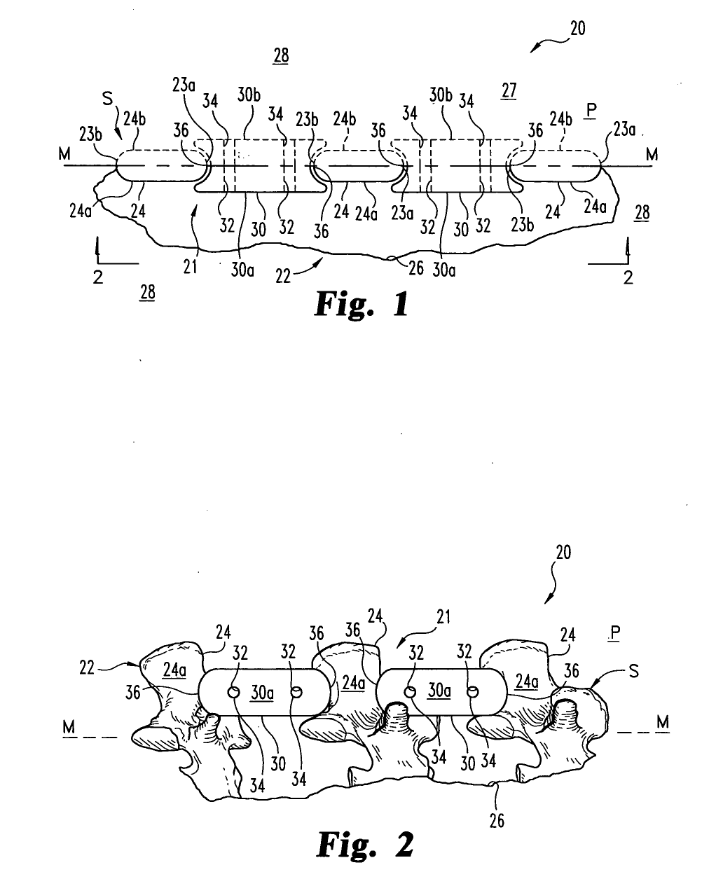

In another embodiment, FIGS. 1 and 2 depict spinal prosthetic system 20 during implantation in spine S of patient P. FIG. 1 is a top view of posterior spinal region 22 of patient P, and FIG. 2 is a side view that corresponds to the view line 2-2 shown in FIG. 1. Median axis M-M is coincident with the median sagittal plane of the body of patient P; where the median sagittal plane is perpendicular to the view plane of FIG. 1 and parallel to the view plane of FIG. 2. System 20 includes prosthetic apparatus 21 configured for implantation in spine S through a posterior approach. Accordingly, posterior spinal region 22 of patient P is shown in the views of FIGS. 1 and 2. For the stage of implantation surgery represented in FIGS. 1 and 2, incision has occurred to define incision site 26. In site 26, several spinous processes 24 are partially exposed along with other subcutaneous tissue of region 22. More specifically, site 26 results from performance of a unilateral surgical procedure to expose a portion of spinous processes 24 of spine S that are generally to lateral one side of axis M-M. The opposite lateral side of axis M-M remains generally intact so that tissue 27 of region 22 is not displaced by incision during the procedure. Spinous processes 24 each have exposed side 24a in site 26 opposite visually hidden side 24b that is at least partially covered by soft tissue 27. It should be understood that tissue 27 includes skin 28 and significant amounts of subcutaneous tissue underlying skin 28 in the vicinity of side 24b of each spinous process 24, which is relatively undisturbed. In this manner, the unilateral surgical procedure illustrated limits the degree of dissection and displacement of tissue that may otherwise result compared to a surgical procedure that more fully exposes spinous processes 24.

System 20 also includes a number of interspinous prosthetic implant devices 30. Each device 30 is inserted between two opposing processes 24 along median axis M-M, and includes opposing end portions 36 shaped with a curvature that is complimentary to the portion of the spinous process 24 engaged thereby. In this engagement, each end portion 36 partially wraps around the corresponding spinous process 24. Each device 30 also includes a plurality of passageways 32 therethrough that extend from side 30a through opposing side 30b. Each side 30a and 30b has a corresponding pair of opposed openings 34 to a corresponding one of passageway 32. Passageways 32 generally extend laterally across axis M-M as positioned in FIGS. 1 and 2. For each device 30, side 30a is generally exposed for the unilateral surgical procedure state represented in FIGS. 1 and 2, while side 30b is generally obscured. In one nonlimiting embodiment, devices 30 are each comprised of a nonmetallic, organic polymer-based material suitable for spinal implantation; however, other metallic and/or nonmetallic compositions can be utilized in other embodiments.

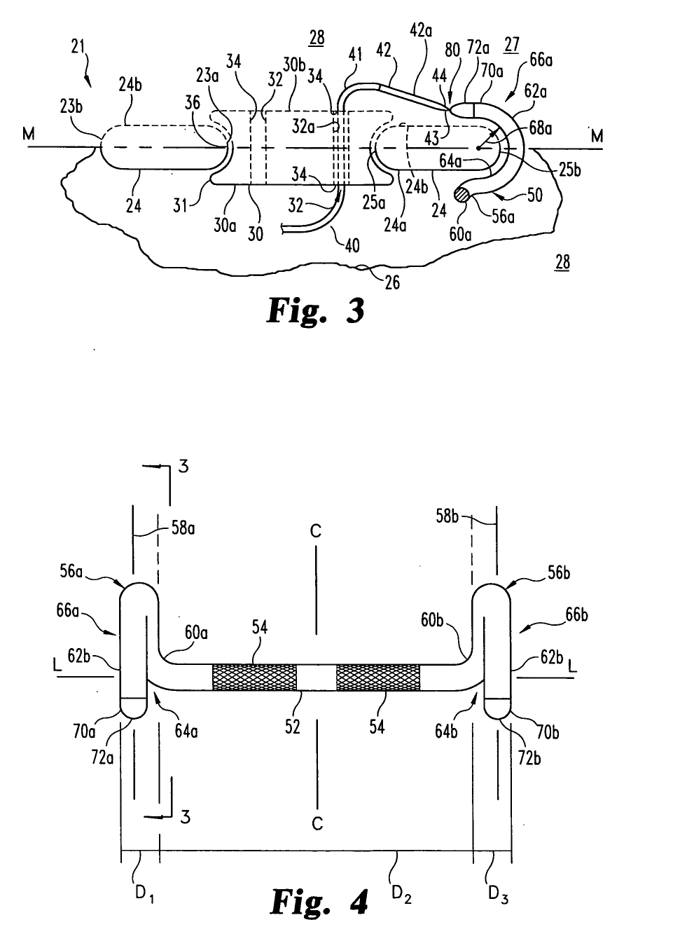

FIG. 3 provides a top view of a portion of prosthetic apparatus 21 in a different state of the unilateral procedure; where like reference numerals refer to like features. In FIG. 3, apparatus 21 includes cable 40, and one of devices 30 is more specifically designed device 31 to enhance clarity of the description with respect to cable 40 as follows. Likewise one of passageways 32 of device 31 is more specifically designated as cable engagement passage 32a. Cable 40 is also alternatively designated as being at least a part of cabling 41.

Cable 40 includes cable end portion 42 with termination 42a. In the illustrated embodiment, termination 43 is structured with a pointed, needle-shaped tip 43 suitable to penetrate soft tissue 27; however, in other embodiments cable 40 may terminate in a differently shaped structure. Cable end portion 42 extends through passage 32a of device 31 from side 30a to side 30b—entering and exiting through the corresponding openings 34. For passage 32a, it should be appreciated that opening 34 on side 30a of device 31 is more readily accessible from the unilaterally exposed area of incision site 26 than the opposite opening 34. After passing through passage 32a, cable end portion 42 extends away from side 30b and is routed through soft tissue 27 next to bone 25 of the adjacent spinous process 24. Bone 25 is further shown with end portion 25a engaged with device 31 and end portion 25b opposite end portion 25a. Tip 43 and cable end portion 42 are configured to dissect and penetrate soft tissue 27, and advance therethrough from end portion 25a towards end portion 25b, wrapping around bone 25. Typically, this cable configuration is desired to further secure device 31; however, at least a portion of cable end portion 42 and/or tip 43 can be hidden from view beneath tissue 27 during the procedure. To assist in the advancement of cable 40 during this blind phase of the procedure, a technique for penetrating and directing end portion 42 of cable 40 through soft tissue T and about bone 25 was discovered—even if end portion 42 and tip 43 are visually hidden by overlying, posterior tissue that may or may not include skin 28.

At least one magnet and/or magnetically attractable material 44 is included in cable 40 to facilitate magnetic coupling with termination 42a. If a magnet is used, it can be one or more different types provided in a configuration suitable for implantation in spine S. If a magnetically-attractable material is used it can also be of any type suitably configured for implantation, such as a paramagnetic alloy including iron (Fe), just to name one nonlimiting example.

Also shown in FIG. 3 is a distal portion of surgical instrument 50 magnetically coupled to tip 43. Referring additionally to FIG. 4, further details concerning surgical instrument 50 are shown. Surgical instrument 50 includes a central, longitudinal portion 52 that has a central axis coincident with longitudinal axis L-L of instrument 50 as shown in FIG. 4. Longitudinal portion 52 further includes knurling 54 along its length to provide a more secure grip for the surgeon. At opposite ends of instrument 50 are end portions 56a and 56b. End portion 56a extends along axis L-L distance D1 which is less than distance D2 of longitudinal portion 52 along axis L-L. End portion 56b extends along axis L-L distance D3 which is also less than distance D2 of longitudinal portion 52. Further, the sum of distances D1 and D3 is also less than distance D2 (D1+D3<D2). It is envisioned that instrument 50 would be made from a material of suitable rigidity and compatibility with human tissues as would be known to those skilled in the art, and further that at least part of instrument 50 has one or more selected magnetic properties as further described hereinafter.

End portion 56a is the same as the distal portion shown in FIG. 3, being separated from the remainder of instrument 50 at the illustrated cross section. This cross section corresponds to section line 3-3 shown in FIG. 4, and reveals a cylindrical, sectional shape; however, the cross-sectional shape and relative sizing may vary in other embodiments. End portion 56a lateral extends away from axis L-L by virtue of turn 60a to terminate in hook-shaped structure 62a. Turn 60a positions hook-shaped structure 62a along imaginary plane 58a that is generally orthogonal to axis L-L, although in other embodiments, a different orientation/geometry may be employed.

Hook-shaped structure 62a includes another turn corresponding to curve 64a. Curve 64a forms hook 65a. Curve 64a corresponds to the bend radius represented by ray 68a shown in FIG. 3. For the embodiment shown, it should be appreciated that curve 64a subtends angle R about bone 25, as demonstrated by the rotation of ray 68a about its origin from instrument tip 70a to turn 60a. In a more preferred arrangement, angle R is at least 90 degrees. In an even more preferred arrangement, angle R is at least 135 degrees. In a most preferred arrangement, angle R is about 180 degrees. It should be appreciated that curve 64a corresponds to a second turn such that end portion 56a includes multiple turns in different directions, including hook 65a, that are collectively designated multiples turns or bends 66a. Further, it should be appreciated that any turn can be of a smooth continuous curve type and/or provided by straight segments that are discontinuously joined together.

As shown in FIG. 4, axis C-C intersects axis L-L perpendicularly at the midpoint of the longitude of instrument 50 along axis L-L. A plane coincident with axis C-C that is orthogonal to the view plane of FIG. 4 serves as a plane of symmetry for device 50 such that end portions 56a and 56b are mirror images of one another. Accordingly, end portion 56b also laterally extends away from longitudinal portion 52 in correspondence to turn 60b. End portion 56b includes hook-shaped structure 62b including curve 64b. Curve 64b forms hook 65b with a bend radius as represented by ray 68b. Ray 68b also can be rotated about its origin to subtend angle R as previously described. End portions 56a and 56b each have instrument tip 70a and 70b, respectively. Instrument tips 70a and 70b each include at least one corresponding magnet 72. Each magnet 72 is of a permanent type structured to magnetically attract and couple to at least a part of cable 40, and is of any variety suitable for use in surgical instrumentation. In one nonlimiting example, magnet 72 includes neodymium (Nd) and provides at least 5 pounds of pulling force when magnetically coupled to an appropriate structure.

In operation, a surgeon uses instrument 50 to selectively direct cable 40 through tissue 27. For the posterior approach illustrated in FIG. 3, end portion 56a is placed proximate to the exposed portion (side 24a) of bone 25, and rotated counterclockwise from the FIG. 3 view to turn hook 65a about bone 25, and direct instrument tip 70a beneath tissue 27. Correspondingly, instrument tip 70a advances in a direction from end portion 25b of bone 25 toward end portion 25a of bone 25. Either before or after this manipulation of instrument 50, end portion 42 of cable 40 is inserted through passage 32a and into tissue 27 as previously described. To assist with the advancement of cable 40 through tissue 27, the counterclockwise rotation of instrument 50 positions instrument tip 70a and magnet 72 so that a magnetic attraction with magnet and/or magnetically attractable material 44 results. Magnet 72 is polarized and oriented relative to magnet and/or magnetically attractable material 44 to result in such attraction.

Based on this magnetic attraction, instrument tip 70a and tip 43 become magnetically coupled, forming magnetic coupling 80. With formation of this magnetic coupling 80, instrument 50 is withdrawn from tissue 27 by clockwise rotation, which pulls cable 40 around bone 25 towards end portion 25b, and routes tip 43 to incision site 26. As tip 43 emerges from tissue 27 proximate to end portion 25b, the surgeon can apply a mechanical force to overcome the magnetic attraction of magnetic coupling 80 to separate instrument 50 and cable 40. Once separated, the surgeon can continue to route and/or secure cable 40 as desired for the particular procedure being performed. Optionally, the procedure may include the separation and removal of tip 43 from the remainder of cable 40 and/or other divisions of cable 40 after routing about bone 25. Alternatively or additionally, additional cabling may be joined to cable 40 with or without tip 43 remaining. Notably, end portion 56b of instrument 50 is structured to perform a like operation about a spinous process 24 at an opposite end portion, such as end portion 25a, with rotation into soft tissue being in a clockwise direction and rotation out of such tissue being counterclockwise.

As an alternative to unilateral interspinous implantation, the techniques of the present application find application in other procedures. By way of nonlimiting example, one alternative embodiment includes: (a) performing a surgical procedure to implant prosthetic apparatus including a cable; (b) utilizing an instrument during this procedure that includes a hook-shaped structure; (c) magnetically coupling the hook-shaped structure of the instrument to an end portion of the cable; and (d) moving the instrument while coupled to the cable to direct the cable through tissue with at least a portion of the cable being visually hidden; and (e) securing the cable after advancement.

Many other embodiments of the present application are also envisioned. For instance, if cable tip 43 is carrying a magnet, then instrument tip 70a or 70b need not carry magnet 72, but instead can be made from a material that is attracted to a magnet to form coupling 80, such as a paramagnetic alloy. Also, it should be appreciated that both cable 40 and instrument 50 can be coupled together with each carrying a magnet oriented so that opposite poles form magnetic coupling 80. In another example, magnet 72 of instrument end portion 56a and magnet 72 of instrument end portion 56b are provided as opposite poles of the same magnet and/or cable 40 is provided in the form of a single magnet with opposite poles at different ends. Alternatively or additionally, a magnet included in instrument 50 and/or cable 40 is provided as an electromagnetic type.

Still another alternative embodiment is a kit that includes cabling, a number of interspinous prosthetic devices 30 of different size, and instrument 50 to assist in directing a cable by selective magnetic coupling. In a further embodiment it is envisioned that instrument 50 may not be symmetric about a plane and/or may have only one hook-shaped structure. In still further embodiments, instrument 50 may include an angled, curved, or approximately straight end portion that does not include a hook on one or more ends and or lacks multiple turns, bends, curves, or angles in different directions or planes. In yet further embodiments, rotational manipulation of instrument 50 may not be used and/or may differ.

While the invention has been illustrated and described in detail in the drawings and foregoing description, the same is to be considered illustrative and not restrictive in character, it being understood that only selected embodiments have been shown and described and that all changes, equivalents, and modifications that come within the scope of the inventions described herein or defined by the following claims are desired to be protected. Any experiments, experimental examples, or experimental results provided herein are intended to be illustrative of the present invention and should not be construed to limit or restrict the invention scope. Further, any theory, mechanism of operation, proof, or finding stated herein is meant to further enhance understanding of the present invention and is not intended to limit the present invention in any way to such theory, mechanism of operation, proof, or finding. In reading the claims, words such as “a”, “an”, “at least on”, and “at least a portion” are not intended to limit the claims to only one item unless specifically stated to the contrary. Further, when the language “at least a portion” and/or “a portion” is used, the claims may include a portion and/or the entire item unless specifically stated to the contrary.

What is claimed is:

1. A method, comprising:
performing surgery to implant a prosthesis between two spinous processes, one of the spinous processes remaining covered by soft tissue throughout the surgery;
magnetically coupling an instrument to a cable;
while the instrument is coupled to the cable, advancing the cable beneath the soft tissue and about the one of the spinous processes, an end portion of the cable being visually hidden during performance of the advancing; and
engaging the cable to the prosthesis.

2. The method of claim 1, wherein the engaging includes routing the cable through a passageway defined by the prosthesis before the advancing.

3. The method of claim 1, wherein the surgery is of a unilateral type that exposes subcutaneous tissue on one side of each of the two spinous processes and leaves another side of each of the two spinous processes at least partially covered by skin.

4. The method of claim 1, which includes:
routing one or more of the cable or different cabling about another of the spinous processes through other soft tissue that remains visually hidden by skin covering the other soft tissue; and
performing at least a portion of the routing with the instrument magnetically coupled to the one or more of the cable or the different cabling.

5. The method of claim 1, wherein the cable terminates with one or more of a magnet or a magnetically attractable material.

6. The method of claim 1, wherein the cable has a tip and further comprising separating the tip after said advancing.

7. The method of claim 1, wherein the instrument includes a first hook-shaped end portion opposite a second hook-shaped end portion and is approximately symmetric about a plane though a longitudinal midpoint.

8. An apparatus, comprising: a surgical instrument to perform a unilateral surgical procedure to implant a prosthetic device between two spinous processes, the instrument including a longitudinal portion extending along a longitudinal axis and a first end portion, the longitudinal portion extending a greater distance along the longitudinal axis than the first end portion, the first end portion including multiple turns in different directions to laterally extend away from the longitudinal axis and provide a hook-shaped structure with a tip, the hooked-shaped structure including one or more of a first magnet and a magnetically attractable material and being shaped and sized to turn about one of the spinous processes at least 90 degrees to magnetically couple with a portion of a cable visually hidden by soft tissue covering one side of the one of the spinous processes.

9. The apparatus of claim 8, wherein the tips includes the first magnet and the instrument includes a second end portion with at least two turns in different directions and another tip including a second magnet.

10. The apparatus of claim 9, wherein the tool is symmetric about a plane coincident with a midpoint of the instrument and approximately perpendicular to the longitudinal axis such that the first end portion and the second end portion are each a mirror image of the other.

11. The apparatus of claim 8, wherein the hook-shaped structure extends along a plane generally orthogonal to the longitudinal axis.

12. The apparatus of claim 8, wherein the instrument includes means for pulling a hidden cable tip about a first portion of a spinous process bone and about a second portion opposite the first portion.

13. A system, comprising:
an interspinous prosthesis device structured to be implanted between two spinous processes of a spine of a patient;
cabling to engage the prosthetic device and tissue of the patient; and
a surgical instrument including a central longitudinal portion that defines a longitudinal axis and a first end portion, the longitudinal portion extending a greater distance along the longitudinal axis of the instrument than the first end portion, the first end portion including a hook-shaped structure that turns away from the longitudinal axis, the hook-shaped termination carrying one or more of a magnet and a magnetically attractable material to magnetically couple to a portion of the cabling when visually hidden by soft tissue covering at least a portion of one of the spinous processes.

14. The system of claim 13, wherein the system is a kit of parts including the interspinous prosthesis, the cabling, and the instrument, and further comprising one or more differently sized interspinous prostheses.

15. The system of claim 13, wherein the instrument includes a second end portion with another hooked-shaped structure, the first end portion and the second end portion each are a mirror image of the other about a plane perpendicular to the longitudinal axis and that is positioned between the first end portion and the second end portion.

16. The system of claim 13, wherein:
the prosthetic device includes means for engaging the cabling; and
the portion of the cabling includes means for magnetically coupling to the first end portion of the instrument.

17. A method, comprising:
performing a surgical procedure with a cable;
utilizing an instrument during the procedure, the instrument including a hook-shaped structure;
magnetically coupling the hook-shaped structure of the instrument to the cable;
while the instrument is coupled to the cable, moving the instrument to advance the cable through tissue while at least an end portion of the cable is visually hidden beneath at least part of the tissue; and
securing the cable after advancement.

18. The method of claim 17, wherein the surgical procedure is of a unilateral type that exposes one lateral side of a spinous process of a patient's spine while skin at least partially covers an opposing lateral side of the spinous process throughout the procedure.

19. The method of claim 18, wherein the hook-shaped structure includes one or more of a magnet or a magnetically attractable material and defines a curvature effective to turn about the spinous process to couple to the end portion of the cable.

20. The method of claim 17, wherein the instrument includes a longitudinal portion extending along a longitudinal axis and an end portion, the longitudinal portion extending a greater distance along the longitudinal axis than the end portion, the end portion including multiple turns in different directions to terminated with a hook-shaped structure.

21. The method of claim 17, which includes routing the cable through a passageway of a prosthetic device.

22. The method of claim 21, wherein the moving routes the cable about a spinous process and the prosthetic device is an interspinous prosthesis.

23. The method of claim 17, wherein the cable terminates with a needle-shaped tip structured to penetrate soft tissue.

24. The method of claim 17, which includes separating the needle-shaped tip from the cable.
